(19) United States
(12) Patent Application Publication
(10) Pub. No.: US 2025/0363634 A1
(43) Pub. Date: Nov. 27, 2025

(54) IMAGE PROCESSING DEVICE, MEDICAL SYSTEM, METHOD OF OPERATING IMAGE PROCESSING DEVICE, AND TRAINING DEVICE

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP)

(73) Assignee: OLYMPUS MEDICAL SYSTEMS CORP., Tokyo (JP)

(21) Appl. No.: 19/290,895

(22) Filed: Aug. 5, 2025

Related U.S. Application Data
(63) Continuation of application No. PCT/JP2023/004406, filed on Feb. 9, 2023.

(51) Int. Cl. G06T 7/00; A61B 5/00; G06T 5/50; G06V 10/60

(52) U.S. Cl. CPC G06T 7/0012; A61B 5/0033; A61B 5/0071; G06T 5/50; G06V 10/60; G06T 2207/10064; G06T 2207/20221; G06T 2207/30101

(57) ABSTRACT

An image processing device includes a processor including hardware, the processor being configured to acquire an imaging signal obtained by capturing an image of fluorescence, generate a fluorescence image based on the imaging signal, extract a first pixel a luminance value of which is at or above a first threshold in the fluorescence image, specify a first area based on positional information on the first pixel, extract a second pixel a luminance value of which is at or under a second threshold in the first area of the fluorescence image, specify a second area that is an insufficient hemostasis area based on positional information on the second pixel, and superimpose the insufficient hemostasis area onto an output image and output the output image with the insufficient hemostasis area being superimposed thereon.

### CROSS REFERENCES TO RELATED APPLICATIONS

This application is a continuation of International Application No. PCT/JP2023/004406, filed on Feb. 9, 2023, the entire contents of which are incorporated herein by reference.

### BACKGROUND

### 1. Technical Field

The present disclosure relates to an image processing device, a medical system, a method of operating an image processing device, and a training device.

### 2. Related Art

In the related art, in the field of medicine, minimally invasive therapy using an endoscope and a laparoscope, or the like, has been performed widely. For example, endoscopic submucosal dissection (ESD) has been performed widely as minimally invasive therapy using an endoscope and a laparoscope.

In ESD, an outer circumference of a lesion of living tissue is cut with an energy device, such as a high-frequency slitter, or the like. When the living tissue is thermally denatured by thermal treatment provided with an energy device, advanced glycation end products (AGEs) are generated. AGEs generate fluorescence when excitation light is applied thereto, which makes it possible to visualize the status of thermal treatment using a fluorescence image (refer to, for example, International Publication Pamphlet No. WO 2020/054723). A practitioner performs hemostasis treatment by heat coagulation on the cut part while observing the fluorescence image.

### SUMMARY

In some embodiments, an image processing device includes a processor including hardware, the processor being configured to acquire an imaging signal obtained by capturing an image of fluorescence, generate a fluorescence image based on the imaging signal, extract a first pixel a luminance value of which is at or above a first threshold in the fluorescence image, specify a first area based on positional information on the first pixel, extract a second pixel a luminance value of which is at or under a second threshold in the first area of the fluorescence image, specify a second area that is an insufficient hemostasis area based on positional information on the second pixel, and superimpose the insufficient hemostasis area onto an output image and output the output image with the insufficient hemostasis area being superimposed thereon.

In some embodiments, provided is a method of operating an image processing device including a processor comprising hardware. The processor is configured to acquire an imaging signal obtained by capturing an image of fluorescence, generate a fluorescence image based on the imaging signal, extract a first pixel a luminance value of which is at or above a first threshold in the fluorescence image, specify a first area based on positional information on the first pixel, extract a second pixel a luminance value of which is at or under a second threshold in the first area of the fluorescence image, specify a second area that is an insufficient hemostasis area based on positional information on the second pixel, and superimpose the insufficient hemostasis area onto an output image and output the output image with the insufficient hemostasis area being superimposed thereon.

In some embodiments, a training device includes a training processor configured to generate a trained model by performing machine learning using teaching data in which a fluorescence image obtained by applying excitation light to living tissue and capturing an image of fluorescence serves as input data and information obtained by superimposing a second area that is an insufficient hemostasis area and that is specified based on positional information on a second pixel a luminance value of which is at or under a second threshold onto the fluorescence image, in a first area that is specified based on a first pixel a luminance value of which is at or above a first threshold in the fluorescence image, serves as output data.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an entire configuration of an endoscope system according to an embodiment;

FIG. 2 is a block diagram illustrating a functional configuration of a relevant part of the endoscope system according to the embodiment;

FIG. 3 is a flowchart illustrating an overview of a process that is executed by a control device;

FIG. 4 is an example of a fluorescence image;

FIG. 5 is a diagram illustrating that a second area is superimposed on the fluorescence image; and

FIG. 6 is a diagram illustrating that the second area is superimposed on a white light image.

### DETAILED DESCRIPTION

An endoscope system including an endoscope having a flexible insertion portion will be described as a mode for carrying out the present disclosure (referred to as “embodiment” below); however, the disclosure is not limited to this and, for example, is applicable to even a rigid scope and an operation robot. The embodiment does not limit the present disclosure. As for illustration of the drawings, the same components are denoted with the same reference numerals and are described. Furthermore, it is necessary to note that the drawings are schematic and the relationship between the thickness and the width of each member, the proportion of each member, etc., are different from actual ones. Components different in size and proportion between the drawings may be contained as well.

### Configuration of Endoscope

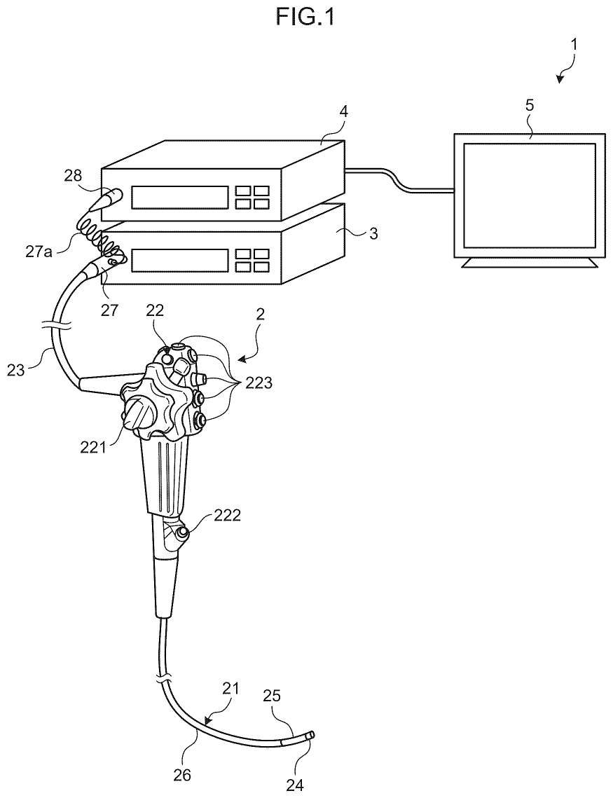

FIG. 1 is a diagram schematically illustrating an entire configuration of an endoscope system according to an embodiment. An endoscope system 1 illustrated in FIG. 1 captures an internal image of the body of a subject, such as a patient, by inserting an insertion portion of an endoscope into a body cavity or a lumen of the subject and displays a display image based on an imaging signal of the captured image on a display device. The endoscope system 1 includes an endoscope 2, a light source device 3, a control device 4 serving as an image processing device, and a display device 5.

### Configuration of Endoscope

First of all, a configuration of the endoscope 2 will be described.

The endoscope 2 generates an imaging signal (RAW data) of a captured internal image of the body of the subject and outputs the generated imaging signal to the control device 4. Specifically, the endoscope 2 generates a first imaging signal obtained by applying white light and capturing an image of return light and a second imaging signal obtained by applying excitation light and capturing an image of fluorescence. The endoscope 2 includes an insertion portion 21, an operation unit 22, and a universal cord 23.

The insertion portion 21 is inserted into the subject. The insertion portion 21 is flexible and elongated. The insertion portion 21 includes a distal end part 24 that incorporates an imaging device to be described below, a curving part 25 that is formed of multiple curving pieces and that flexibly curves, and a flexible tube 26 that is flexible and elongated and that is connected to a proximal end side of the curving part 25.

The distal end part 24 is formed using glass fibers, or the like. The distal end part 24 forms a light guide path for illumination light that is supplied from the control device 4 via the universal cord 23 and the operation unit 22, generates an imaging signal of a captured image of return light of the illumination light, and outputs the imaging signal to the control device 4.

The operation unit 22 includes a curving knob 221 that causes the curving unit 25 to curve in up and down directions and left and right directions, a treatment tool insertion portion 222 into which a treatment tool is inserted, and a plurality of switches 223 serving as an operation input unit that, in addition to the control device 4, inputs operation instruction signals to peripherals, such as an air supply unit, a water supply unit and a gas supply unit, a pre-freeze signal of an instruction for the endoscope system 1 to capture a still image, or a switch signal that switches an observation mode of the endoscope system 1. The treatment tool that is inserted from the treatment tool insertion portion 222 goes out of an opening via a treatment tool channel of the distal end part 24.

The universal cord 23 incorporates at least a light guide and an assembly cable including a single or a plurality of cables. The assembly cable includes a signal line for transmitting and receiving a signal between the endoscope 2 and the control device 4 and for transmitting and receiving the imaging signal (RAW data) and a signal line for transmitting and receiving a drive timing signal (a synchronization signal and a clock signal) for driving the imaging device to be described below. The universal cord 23 includes a connector 27 that is detachable from the control device 4 and a connector 28 to which a coiled coil cable 27a extends and that is detachable from the control device 4 at an end of extension of the coil cable 27a.

### Configuration of Light Source Device

A configuration of the light source device will be described next.

The light source device 3 applies white light and excitation light as the illumination light to the living tissue. One end of the light guide of the endoscope 2 is connected to the light source device 3 and, under the control of the control device 4, the light source device 3 supplies the illumination light to be applied to the inside of the subject to the one end of the light guide. The light source device 3 is realized using at least one of light sources that are a light emitting diode (LED) light source, a xenon lamp, and a semiconductor laser device, such as a laser diode (LD), a processor that is a processing device including hardware, such as a field programmable gate array (FPGA) or a central processing unit (CPU), and a memory that is a temporary storage area that the processor uses. Note that the light source device 3 and the control device 4 may be configured to communicate individually as illustrated in FIG. 1 or may be configured integrally.

### Configuration of Control Device

A configuration of the control device 4 will be described next.

The control device 4 controls each unit of the endoscope system 1. The control device 4 controls the light source device 3 and thereby supplies illumination light to be applied to the subject by the endoscope 2. The control device 4 performs various types of image processing on the imaging signal that is input from the endoscope 2 and outputs the processed imaging signal to the display device 5.

### Configuration of Display Device

A configuration of the display device 5 will be described next.

The display device 5 displays a display image based on a video signal that is input from the control device 4 under the control of the control device 4. The display device 5 is realized using a display panel of organic electro luminescence (EL), liquid crystals, or the like.

### Functional Configuration of Relevant Part of Endoscope System

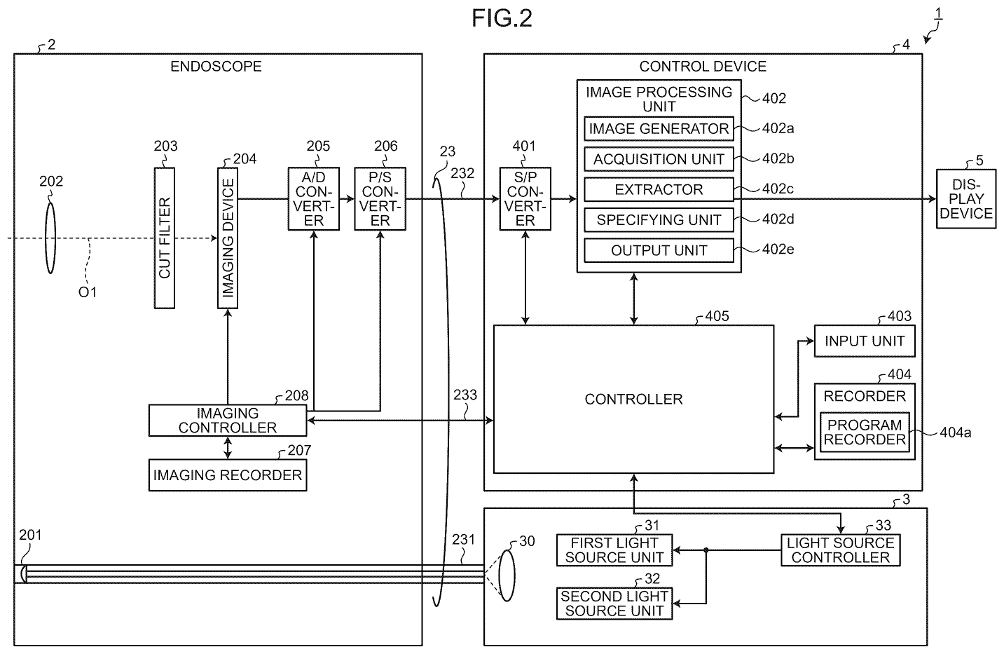

A functional configuration of a relevant part of the endoscope system 1 described above will be described next. FIG. 2 is a block diagram illustrating the functional configuration of the relevant part of the endoscope system 1.

### Configuration of Endoscope

First of all, a configuration of the endoscope 2 will be described.

The endoscope 2 includes an illuminating optical system 201, an imaging optical system 202, a cut filter 203, an imaging device 204, an A/D converter 205, a P/S converter 206, an imaging recorder 207, and an imaging controller 208. Note that each of the illuminating optical system 201, the imaging optical system 202, the cut filter 203, the imaging device 204, the A/D converter 205, the P/S converter 206, the imaging recorder 207, and the imaging controller 208 is arranged in the distal end part 24.

The illuminating optical system 201 applies the illumination light that is supplied from a light guide 231 that is formed of optical fibers, and the like, to the subject (living tissue). The illuminating optical system 201 is realized using a single lens, a plurality of lenses, or the like.

The imaging optical system 202 focuses light, such as reflection light that is reflected from the subject, return light from the subject, or fluorescence that the subject emits, thereby forming a subject image (ray of light) on a light receiving surface of the imaging device 204. The imaging optical system 202 is realized using a single lens, a plurality of lenses, or the like.

The cut filter 203 is arranged on an optical axis O1 of the imaging optical system 202 and the imaging device 204. The cut filter 203 blocks light having a wavelength band of reflection light or return light of the excitation light that is supplied from the light source device 3 and that is from the subject and transmits light of a wavelength band on a side of wavelengths longer than those of the excitation light.

Under the control of the imaging controller 208, the imaging device 204 receives the subject image (ray of light) that is formed by the imaging optical system 202 and that is transmitted through the cut filter 203, performs photoelectric conversion to generate an imaging signal (RAW data), and outputs the imaging signal to the A/D converter 205. The imaging device 204 is realized using a charge coupled device (CCD) or complementary metal oxide semiconductor (CMOS) image sensor that is formed by arranging any one of color filters forming a Bayer array (RGGB) in each of a plurality of pixels arranged in a two-dimensional matrix.

Under the control of the imaging controller 208, the A/D converter 205 performs the A/D conversion processing on the analog imaging signal that is input from the imaging device 204 and outputs the processed imaging signal to the P/S converter 206. The A/D converter 205 is realized using an A/D conversion circuit, or the like.

Under the control of the imaging controller 208, the P/S converter 206 performs parallel/serial conversion on the digital imaging signal that is input from the A/D converter 205 and outputs the imaging signal on which the parallel/serial conversion is performed to the control device 4 via a first transmission cable 232. The P/S converter 206 is realized using the P/S conversion circuit, or the like. Note that, according to the first embodiment, an E/O converter that converts an imaging signal into an optical signal may be provided instead of the P/S converter 206 and the imaging signal may be output to the control device 4 using an optical signal and may be transmitted to the control device 4 by wireless communication according to, for example, Wi-Fi (Wireless Fidelity) (trademark).

The imaging recorder 207 records various types of information on the endoscope 2 (for example, pixel information on the imaging device 204 and the characteristics of the cut filter 203). The imaging recorder 207 records various types of setting data and parameters for control that are transmitted from the control device 4 via a second transmission cable 233. The imaging recorder 207 is configured using a non-volatile memory or a volatile memory.

The imaging controller 208 controls operations of each of the imaging device 204, the A/D converter 205, and the P/S converter 206 based on the setting data that is received from the control device 4 via the second transmission cable 233. The imaging controller 208 is realized using a time generator (TG), a processor that is a processing device including hardware, such as a CPU, and a memory that is a temporary storage area that the processor uses.

### Configuration of Light Source Device

A configuration of the light source device 3 will be described next.

The light source device 3 includes a condenser lens 30, a first light source unit 31, a second light source unit 32, a light source controller 33.

The condenser lens 30 focuses light that is emitted by each of the first light source unit 31 and the second light source unit 32 and emits the light to the light guide 231. The condenser lens 30 is configured using a single lens or a plurality of lenses.

Under the control of the light source controller 33, the first light source unit 31 emits white light (normal light) that is visible light and thereby supplies white light to the light guide 231. The first light source unit 31 is configured using a collimating lens, a white LED lamp, a driver, etc. The first light source unit 31 may simultaneously emit light with a red LED lamp, a green LED lamp, and a blue LED lamp, thereby supplying white light that is visible light. Needless to say, the first light source unit 31 may be configured using a halogen lamp, a xenon lamp, or the like.

Under the control of the light source controller 33, the second light source unit 32 emits excitation light having a given wavelength band and thereby supplies the excitation light as the illumination light to the light guide 231. The excitation light is a wavelength that excites a substance, such as advanced glycation end products (AGEs) that the thermally denatured area contains, and has, for example, a wavelength band between 400 nanometers (nm) and 430 nm inclusive (the center wavelength is 415 nm). The thermally denatured area is an area where heat treatment is performed with an energy device, such a high-frequency slitter, and accordingly living tissue is denatured by heat. The excitation light that is applied by the second light source unit 32 is blocked by the cut filter 203 and the fluorescence (whose wavelength is 540 nm) that is generated from the AGEs is transmitted through the cut filter 203 and thus it is possible to capture a fluorescence image. The second light source unit 32 is realized using a collimating lens, a semiconductor laser, such as a violet laser diode (LD), a driver, etc.

The light source controller 33 is configured using a processor that is a processing device including hardware, such as a field programmable agate array (FPGA) or a CPU, and a memory that is a temporary storage area that the processor uses. The light source controller 33 controls light emission timing, the light emission intensity, the light emission time, etc., of each of the first light source unit 31 and the second light source unit 32.

### Configuration of Control Device

The control device 4 includes a S/P converter 401, an image processing unit 402, an input unit 403, a recorder 404, and a controller 405.

Under the control of the controller 405, the S/P converter 401 performs serial/parallel conversion on the imaging signal that is received from the endoscope 2 via the first transmission cable 232 and outputs the processed imaging signal to the image processing unit 402. Note that, in the case where the endoscope 2 outputs the imaging signal in an optical signal, an O/E converter that converts the optical signal into an electric signal may be provided instead of the S/P converter 401. In the case where the endoscope 2 transmits the imaging signal by wireless communication, a communication module capable of receiving a radio signal may be provided instead of the S/P converter 401.

The image processing unit 402 is realized using a processor including hardware, such as a CPU, a graphics processing unit (GPU) or a FPGA, and a memory that is a temporary storage area that the processor uses. Under the control of the controller 405, the image processing unit 402 performs given image processing on the imaging signal that is input from the S/P converter 401 and outputs the processed imaging signal to the display device 5. The image processing unit 402 generates a white light image from the first imaging signal and generates a fluorescence image from the second imaging signal. The image processing unit 402 includes an image generator 402a, an acquisition unit 402b, an extractor 402c, a specifying unit 402d, and an output unit 402e.

The image generator 402a generates a white light image from a first imaging signal obtained by applying white light from the first light source unit 31 to living tissue and capturing an image of return light. The image generator 402a generates fluorescence image from a second imaging signal obtained by applying fluorescence light from the second light source unit 32 to living tissue and capturing an image of the fluorescence.

The acquisition unit 402b acquires the white light image and the fluorescence image from the image generator 402a. The acquisition unit 402b acquires the first imaging signal and the second imaging signal from the endoscope 2.

The extractor 402c extracts a first pixel whose luminance value is at or above a first threshold in the fluorescence image. The extractor 402c also extracts a second pixel whose luminance value is at or under a second threshold in a first area of the fluorescence image. Note that the first threshold is larger than the second threshold.

Based on positional information on first pixels, the specifying unit 402d specifies the first area as a circular area that is formed by connecting the first pixels. Based on positional information on the second pixel, the specifying unit 402d specifies a second area.

The output unit 402e outputs information obtained by superimposing the first area and the second area on the fluorescence image. The output unit 402e also outputs information obtained by superimposing the first area and the second area on the white light image.

The input unit 403 receives inputs of various types of operations on the endoscope system 1 and outputs the received operations to the controller 405. The input unit 403 is configured using a mouse, a foot switch, a keyboard, a button, a switch, a touch panel, etc.

The recorder 404 is realized using a volatile memory, a non-volatile memory, a solid-state drive (SSD) or a hard disk drive (HDD), or a recording medium, such as a memory card. The recorder 404 records data containing various types of parameters necessary for operations of the endoscope system 1. The recorder 404, for example, stores positional information on the first pixel and the second pixel, positional information on the first area and the second area, etc. The recorder 404 includes a program recorder 404a that records various types of programs for running the endoscope system 1.

The controller 405 is realized using a processor including hardware, such as a FPGA or a CPU, and a memory that is a temporary storage area that the processor uses. The controller 405 generally controls each of the units forming the endoscope system 1.

Process performed by Control Device

A process that the control device 4 executes will be described next.

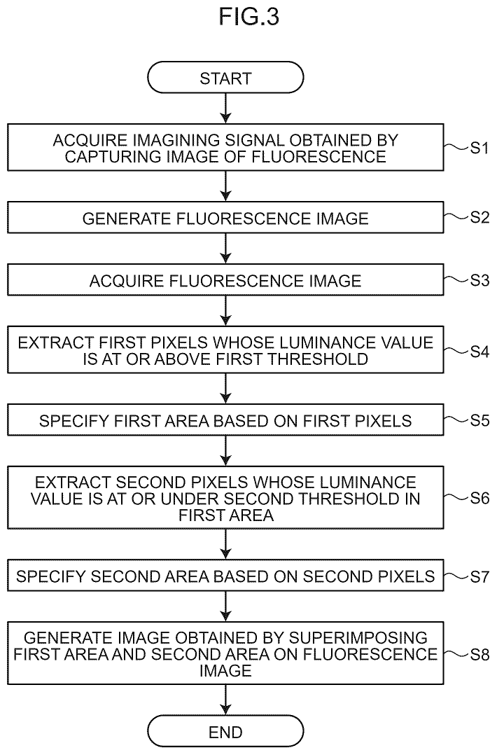

FIG. 3 is a flowchart illustrating an overview of the process that the control device executes. As illustrated in FIG. 3, first of all, the acquisition unit 402b acquires a second imaging signal obtained by applying excitation light from the second light source unit 32 to living tissue and capturing an image of fluorescence (step S1).

Subsequently, the image generator 402a generates a fluorescence image from the second imaging signal that the acquisition unit 402b acquires (step S2). The fluorescence image that is generated by the image generator 402a is stored in the recorder 404.

Thereafter, the acquisition unit 402b acquires the fluorescence image from the recorder 404 (step S3). Note that the acquisition unit 402b may acquire a fluorescence image from an external server via an Internet line.

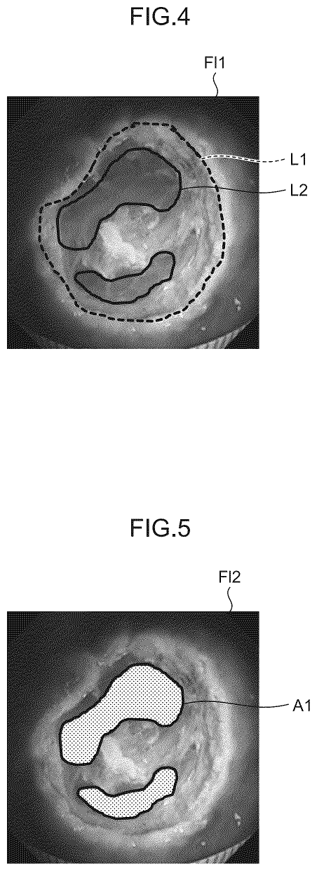

FIG. 4 is an example of the fluorescence image. A fluorescence image FI1 illustrated in FIG. 4 is an image obtained by capturing an image of fluorescence that a thermally denatured area where living tissue is thermally denatured generates. The thermally denatured area is formed by performing thermal processing on living tissue with an energy device and contains AGEs (advanced glycation end products). In ESD, an outer circumference of a lesion of the living tissue is cut with an energy device, such as a high-frequency slitter. It is presented that, because AGEs generate fluorescence when excitation light is applied thereto, a portion corresponding to the outer circumference of the lesion in the fluorescence image FIL is white approximately circularly.

Back to FIG. 3, the extractor 402c extracts first pixels whose luminance value is at or above the first threshold in the fluorescence image FIL (step S4). Accordingly, it is possible to extract a portion that has a high luminance value in the fluorescence image FI1 and that has been cut with the energy device.

Furthermore, based on positional information on the first pixels, the specifying unit 402d specifies a first area as a circular area that is formed by connecting the first pixels (step S5). Connecting parts with a high luminance value in the fluorescence image FI1 makes it possible to specify a circular first area presented in a dotted line L1. Note that the first area is the whole inner side of the dotted line L1.

Subsequently, the extractor 402c extracts second pixels whose luminance value is at or under the second threshold in the first area in the fluorescence image FI1 (step S6). Accordingly, it is possible to extract a portion with a low luminance value where thermal processing is insufficient in the fluorescence image FI1.

Furthermore, based on positional information on the second pixels, the specifying unit 402d specifies a second area (step S7). It is possible to specify a second area with a low luminance value that is presented in a solid line L2 in the fluorescence image FI1.

The output unit 402e outputs information obtained by superimposing the first area and the second area on the fluorescence image FI1 (step S8). As a result, an image that is obtained by superimposing the dotted line L1 presenting the first area and the solid line L2 presenting the second area are superimposed on the fluorescence image FI1 and that is illustrated in FIG. 4 is displayed on the display device 5.

According to the embodiment described above, because the solid line L2 presenting the second area is superimposed on the fluorescence image FI1, a practitioner is able to recognize an area where hemostasis treatment is insufficient.

Note that, in ESD, when the practitioner press a given button after cutting a lesion, the display on the display device 5 switches from the white light image, which allows the practitioner to observe the fluorescence image FI1 with the solid line L2 presenting the second area superimposed thereon in FIG. 4. The practitioner performs the hemostasis treatment with an energy device while checking the state of the second area. As a result, it is possible to prevent insufficient hemostasis treatment.

### Modification

FIG. 5 is a diagram illustrating that a second area is superimposed on a fluorescence image. As illustrated in FIG. 5, a second area A1 may be superimposed on a fluorescence image FI2 such that the second area A1 is painted all in a specific color in the fluorescence image FI2. Accordingly, the second area is observed easily.

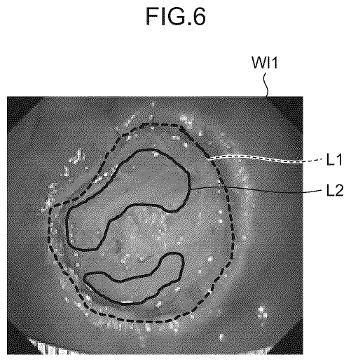

FIG. 6 is a diagram illustrating that a second area is superimposed on a white light image. As illustrated in FIG. 6, the dotted line L1 presenting the first area and the solid line L2 presenting the second area may be superimposed on a white light image WI1. Accordingly, it is possible to see an area where hemostasis is insufficient while observing the white light image.

The fluorescence image FIL presented by FIG. 4 and the white light image presented by FIG. 6 may be displayed side by side and may be observed simultaneously.

The controller 405 may have a function serving as a training unit of a training device of the control device 4. The controller 405 may generate a trained model by performing machine learning using teaching data in which a fluorescence image obtained by applying excitation light to living tissue and capturing an image of fluorescence serves as input data and information obtained by superimposing a second area that is specified based on positional information on second pixels whose luminance value is at or under a second threshold onto the fluorescence image serves as output data, in a first area that is specified based on first pixels whose luminance value is at or above a first threshold in the fluorescence image. The trained model is formed of a neural network in which each layer has a single or a plurality of nodes. The type of machine learning is not particularly limited and, for example, teaching data obtained by associating a plurality of fluorescence images of a subject and images obtained by superimposing a second area that is specified from the fluorescence images onto the fluorescence images may be associated with each other and training data may be prepared, the teaching data and the training data may be input to a computation model based on the multi-layer neural network, and training may be performed. Furthermore, for example, a method based on a deep neural network (DNN) of a multi-layer neural network, such as CNN (Convolutional Neural Network) or 3D-CNN, is used as a method of machine learning. Furthermore, a method based on a recurrent neural network (RNN), LSTM (long short-term memory units) that is an extension of the RNN, or the like, may be used as a method of machine training. Note that a training unit of a training device different from the control device 4 may execute these functions.

According to the present disclosure, it is possible to realize an image processing device, a medical system, a method of operating an image processing device, and a training device that enable easy recognition of an area where hemostasis treatment is insufficient.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device comprising a processor comprising hardware, the processor being configured to
acquire an imaging signal obtained by capturing an image of fluorescence,
generate a fluorescence image based on the imaging signal,
extract a first pixel a luminance value of which is at or above a first threshold in the fluorescence image,
specify a first area based on positional information on the first pixel,
extract a second pixel a luminance value of which is at or under a second threshold in the first area of the fluorescence image,
specify a second area that is an insufficient hemostasis area based on positional information on the second pixel, and
superimpose the insufficient hemostasis area onto an output image and output the output image with the insufficient hemostasis area being superimposed thereon.

2. The image processing device according to claim 1, wherein the first threshold is larger than the second threshold.

3. The image processing device according to claim 1, wherein the fluorescence image is an image obtained by capturing the fluorescence that is generated from a thermally denatured area generated by thermal denaturation of the living tissue.

4. The image processing device according to claim 3, wherein the thermally denatured area is formed by performing thermal processing on the living tissue with an energy device.

5. The image processing device according to claim 1, wherein the first threshold and the second threshold are set according to a luminance value of the fluorescence that is generated from glycation end products that are generated by thermal denaturation of the living tissue.

6. The image processing device according to claim 1, wherein the processor is further configured to output information obtained by superimposing the first area onto the fluorescence image.

7. The image processing device according to claim 1, wherein the processor is further configured to output information obtained by superimposing the second area on a white light image.

8. The image processing device according to claim 1, wherein the processor is further configured to output information obtained by superimposing the first area on a white light image.

9. The image processing device according to claim 1, wherein the processor is further configured to specify, as the first area, a circular area that is formed by connecting first pixels.

10. The image processing device according to claim 1, wherein an inside of the first area is an area that has been cut with an energy device.

11. A method of operating an image processing device comprising a processor comprising hardware, the processor being configured to
acquire an imaging signal obtained by capturing an image of fluorescence,
generate a fluorescence image based on the imaging signal,
extract a first pixel a luminance value of which is at or above a first threshold in the fluorescence image,
specify a first area based on positional information on the first pixel,
extract a second pixel a luminance value of which is at or under a second threshold in the first area of the fluorescence image,
specify a second area that is an insufficient hemostasis area based on positional information on the second pixel, and
superimpose the insufficient hemostasis area onto an output image and output the output image with the insufficient hemostasis area being superimposed thereon.

12. A training device comprising a training processor configured to generate a trained model by performing machine learning using teaching data in which a fluorescence image obtained by applying excitation light to living tissue and capturing an image of fluorescence serves as input data and information obtained by superimposing a second area that is an insufficient hemostasis area and that is specified based on positional information on a second pixel a luminance value of which is at or under a second threshold onto the fluorescence image, in a first area that is specified based on a first pixel a luminance value of which is at or above a first threshold in the fluorescence image, serves as output data.
